(19) United States
(12) Patent Application Publication
(10) Pub. No.: US 2026/0224217 A1
(43) Pub. Date: Aug. 6, 2026

(54) STAPLING DEVICE WITH FIRING LOCKOUT ASSEMBLY

(71) Applicant: Covidien LP, Mansfield, MA (US)

(72) Inventor: Jacob C. BARIL, Norwalk, CT (US)

(21) Appl. No.: 19/158,326

(22) PCT Filed: Feb. 20, 2024
(86) PCT No.: PCT/IB2024/051603

§ 371 Date: Aug. 20, 2025

Related U.S. Application Data
(60) Provisional application No. 63/447,938, filed on Feb. 24, 2023.

(51) Int. Cl. A61B 17/072

(52) U.S. Cl. CPC A61B 17/07207; A61B 2017/07271; A61B 2017/07278; A61B 2017/07285

(57) ABSTRACT

A surgical stapling device includes a staple cartridge having a cartridge body, staples, an actuation sled assembly, and a locking member. The actuation sled assembly includes an actuation sled and a knife that is pivotably supported on the actuation sled and movable between a lowered position and a raised position. The locking member is movable between a locked position in which the locking member retains the actuation sled assembly in a sled retracted position and an unlocked position in which the actuation sled assembly can move to a sled advanced position. The knife is positioned to move the locking member from the locked position to the unlocked position as the knife moves from the lowered position to the raised position.

This Application claims priority from U.S. Provisional Patent Application 63/447,938, filed 24 Feb. 2023, the entire content of which is incorporated herein by reference.

### FIELD

This technology is generally related to surgical stapling devices, and more particularly, to surgical stapling devices including firing lockout assemblies.

### BACKGROUND

Surgical stapling devices configured for endoscopic use are commonly used during surgical procedures to minimize patient trauma and reduce patient recovery times. Typically, endoscopic stapling devices include a tool assembly and a drive assembly that is movable in relation to the tool assembly to actuate the tool assembly. Typically, the drive assembly includes a knife bar having a cutting blade for cutting tissue. The tool assembly includes anvil and cartridge assemblies that are coupled to each other by a pivot member and movable in relation to each other between unclamped and clamped positions in response to movement of the drive assembly from a retracted position towards an advanced position. The cartridge assembly includes a staple cartridge that includes a cartridge body that supports staples and an actuation sled that is movable through the cartridge body in response to movement of the drive assembly from the retracted position to the advanced position to eject the staples from the cartridge body.

Some stapling devices include a staple cartridge that can be replaced after each firing of the stapling device to facilitate reuse of the stapling device. To ensure that a stapling device is not fired with a spent staple cartridge, i.e., a staple cartridge having no staples, or without a staple cartridge, some stapling devices include firing lockout assemblies.

A continuing need exists for a surgical stapling device having a cost-effective assembly for preventing firing of the stapling device when the stapling device includes a spent staple cartridge, a defective staple cartridge, or is missing a staple cartridge entirely.

### SUMMARY

Aspects of this disclosure are directed to a surgical stapling device having a staple cartridge including a cartridge body, staples, an actuation sled assembly, and a locking member. The actuation sled assembly includes an actuation sled and a knife that is pivotably supported on the actuation sled and movable between a lowered position and a raised position. A staple guard supports the locking member that is movable from a locked position in which the locking member retains the actuation sled assembly in a sled retracted position and an unlocked position in which the actuation sled assembly can move to a sled advanced position. The knife is positioned to move the locking member from the locked position to the unlocked position as the knife moves to the raised position.

Aspects of the disclosure are directed to a tool assembly including an anvil and a cartridge assembly having a channel and a staple cartridge. The channel defines a cavity, and the staple cartridge is releasably supported within the cavity and includes a cartridge body, staples, an actuation sled assembly, and a locking member. The cartridge body has a tissue engaging surface and defines a knife slot and staple receiving pockets that extend through the tissue engaging surface. The staples are received within the staple receiving pockets of the cartridge body. The actuation sled assembly is supported within the cartridge body and includes an actuation sled and a knife. The knife is pivotably supported on the actuation sled by a pivot member and is movable between a lowered position and a raised position. The actuation sled assembly is movable within the cartridge body from a sled retracted position to a sled advanced position to eject the staples from the cartridge body and cut tissue clamped between the anvil and the cartridge assembly. The locking member is movable between a locked position and an unlocked position. In the locked position, the locking member is engaged with the actuation sled assembly when the actuation sled assembly is in the sled retracted position to retain the actuation sled assembly in the sled retracted position. The locking member is movable from the locked position to the unlocked a position in response to movement of the knife from the lowered position to the raised position.

Other aspects of the disclosure are directed to a stapling device including a handle assembly, an adapter assembly, and a tool assembly. The adapter assembly has a distal portion and a proximal portion, and the proximal portion of the adapter assembly is coupled to the handle assembly. The tool assembly is coupled to the distal portion of the adapter assembly and includes an anvil and a cartridge assembly. The cartridge assembly includes a channel that defines a cavity and a staple cartridge that is releasably supported within the cavity. The staple cartridge includes a cartridge body, staples, an actuation sled assembly, and a locking member. The cartridge body has a tissue engaging surface and defines a knife slot and staple receiving pockets that extend through the tissue engaging surface. The staples are received within the staple receiving pockets of the cartridge body. The actuation sled assembly is supported within the cartridge body and includes an actuation sled and a knife that is pivotably supported on the actuation sled by a pivot member. The knife is movable between a lowered position and a raised position, and the actuation sled assembly is movable within the cartridge body from a sled retracted position to a sled advanced position to eject the staples from the cartridge body. The locking member is movable between a locked position and an unlocked position. In the locked position, the locking member is engaged with the actuation sled assembly when the actuation sled assembly is in the sled retracted position to retain the actuation sled assembly in the sled retracted position. The locking member is movable from the locked position to the unlocked a position in response to movement of the knife from the lowered position to the raised position.

Other aspects of the disclosure are directed to a tool assembly that includes an anvil and a cartridge assembly. The cartridge assembly includes a channel that defines a cavity and a staple cartridge that is releasably supported within the cavity and includes a cartridge body, staples, and an actuation sled assembly. The cartridge body has a tissue engaging surface and defines a knife slot and staple receiving pockets that extend through the tissue engaging surface. The staples are received within the staple receiving pockets of the cartridge body. The actuation sled assembly is supported within the cartridge body and includes an actuation sled and a knife that is pivotably supported on the actuation sled by a pivot member. The actuation sled assembly is movable between a lowered position and a raised position and is movable within the cartridge body from a sled retracted position to a sled advanced position to eject the staples from the cartridge body. The actuation sled supports a retention member that extends from a proximal portion of the actuation sled and includes spaced resilient arms. Each of the spaced resilient arms has a protrusion that is positioned to engage the knife when the knife is in the lowered position to retain the knife in the lowered position.

In aspects of the disclosure, the tool assembly includes a drive assembly having a working member with a first beam, a second beam, and a vertical strut connecting the first beam to the second beam, and the drive assembly is movable between a drive retracted position and a drive advanced position.

In some aspects of the disclosure, the vertical strut defines a cam slot.

In certain aspects of the disclosure, the knife includes a cam member that is received within the cam slot when the drive assembly is moved towards the drive advanced position to pivot the knife from the lowered position to the raised position.

In aspects of the disclosure, the knife includes a cutting edge that is positioned proximally of the pivot member and an engagement member that is positioned distally of the pivot member.

In some aspects of the disclosure, the engagement member is positioned to engage the locking member when the knife is in the raised position to move the locking member to the unlocked position.

In certain aspects of the disclosure, the locking member includes first resilient tabs that are positioned to prevent advancement of the drive assembly from the drive retracted position to the drive advanced position when the actuation sled assembly is in the sled advanced position.

In aspects of the disclosure, the actuation sled of the actuation sled assembly includes a base portion that defines an opening, and the first resilient tabs are received within the opening in the locked position.

In some aspects of the disclosure, the staple cartridge includes a staple guard that is secured to a bottom wall of the cartridge body to retain the staples within the cartridge body, and the staple guard has a bottom wall that defines a knife slot that is aligned with the knife slot of the cartridge body.

In certain aspects of the disclosure, the first resilient tabs extend in cantilevered fashion from the bottom wall of the staple guard.

In aspects of the disclosure, the actuation sled defines a recess, and the staple guard includes a second resilient tab that is positioned on a distal portion of the bottom wall of the staple guard, and the second resilient tab is positioned to be received in the recess of the actuation sled to retain the actuation sled assembly in the sled advanced position.

In some aspects of the disclosure, the engagement member is formed on a distal end of the knife and has a T-shaped configuration.

In certain aspects of the disclosure, the cam slot of the vertical strut of the working member has a J-shaped configuration.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of the disclosure with a tool assembly of the stapling device in an unclamped, pre-fired position;

FIG. 2 is side perspective view of the tool assembly of the stapling device shown in FIG. 1 with a staple cartridge of a cartridge assembly of the surgical stapling device separated from a channel of the cartridge assembly;

FIG. 3 is an exploded view of a staple cartridge of the cartridge assembly shown in FIG. 2;

FIG. 3A is a perspective view from above of a staple guard of the staple cartridge shown in FIG. 3;

FIG. 4 is a side perspective, cutaway view of an actuation sled assembly of the staple cartridge shown in FIG. 3 and a drive assembly of the stapling device shown in FIG. 1 in a pre-fired, clamped position;

FIG. 4A is a side perspective view of the actuation sled assembly shown in FIG. 4 taken from the opposite side of the actuation sled assembly;

FIG. 5 is a cross-sectional view taken along a longitudinal axis of the tool assembly of the stapling device shown in FIG. 1 in a clamped, pre-fired position;

FIG. 6 is a cross-sectional, cutaway view taken along the longitudinal axis of the tool assembly of the stapling device shown in FIG. 5 as the drive assembly is advanced from the clamped, pre-fired position towards a drive advanced position during a firing stroke of the stapling device to engage a knife of the actuation sled assembly to pivot the knife from a lowered position towards a raised position;

FIG. 7 is a cross-sectional, cutaway view taken along the longitudinal axis of the tool assembly of the stapling device shown in FIG. 6 as the drive assembly is advanced during the firing stroke of the stapling device to move the knife of the actuation sled assembly from the lowered position to the raised position;

FIG. 8 is a cross-sectional, cutaway view taken along the longitudinal axis of the tool assembly of the stapling device shown in FIG. 7 as the drive assembly is advanced further during the firing stroke of the stapling device to position the knife in the raised position and release an actuation sled assembly retaining member;

FIG. 9 is a cross-sectional, cutaway view taken along the longitudinal axis of the tool assembly of the stapling device shown in FIG. 8 with the drive assembly in the drive advanced position during the firing stroke of the stapling device with the knife in the raised position and the actuation sled assembly retained in a sled advanced position;

FIG. 10 is a cross-sectional, cutaway view taken along the longitudinal axis of the tool assembly of the stapling device shown in FIG. 9 as the drive assembly moves from the drive advanced position towards a drive retracted position during the firing stroke of the stapling device as the knife is moved to the lowered position;

FIG. 11 is a cross-sectional, cutaway view taken along the longitudinal axis of the tool assembly of the stapling device shown in FIG. 10 as the drive assembly moves from the drive advanced position towards a drive retracted position during the firing stroke of the stapling device with the knife in the lowered position;

FIG. 12 is a side perspective view of an alternate version of the actuation sled assembly of the stapling device shown in FIG. 1 with the knife in the lowered position;

FIG. 13 is a cross-sectional view taken along section line 13-13 of FIG. 12;

FIG. 14 is a cross-sectional view taken along section line 14-14 of FIG. 12;

FIG. 15 is a cross-sectional, cutaway view taken along the longitudinal axis of the tool assembly of the stapling device shown in FIG. 1 with the actuation sled assembly shown in FIG. 12 in the clamped, pre-fired position;

FIG. 16 is a cross-sectional, cutaway view taken along the longitudinal axis of the tool assembly of the stapling device shown in FIG. 1 with the actuation sled assembly shown in FIG. 12 as the drive assembly is advanced from the clamped, pre-fired position towards a drive advanced position during a firing stroke of the stapling device to engage the knife of the actuation sled assembly to pivot the knife from a lowered position towards a raised position;

FIG. 17 is a cross-sectional view taken along section line 17-17 of FIG. 16;

FIG. 18 is a cross-sectional, cutaway view taken along the longitudinal axis of the tool assembly of the stapling device shown in FIG. 1 with the actuation sled assembly shown in FIG. 12 as the drive assembly is advanced towards the drive advanced position during a firing stroke of the stapling device to position the knife in the raised position; and

FIG. 19 is a cross-sectional, cutaway view taken along the longitudinal axis of the tool assembly of the stapling device shown in FIG. 18 as the drive assembly moves from the drive advanced position towards the drive retracted position during the firing stroke of the stapling device with the knife in the lowered position.

### DETAILED DESCRIPTION

The disclosed surgical stapling device will now be described in detail with reference to the drawings in which like reference numerals designate identical or corresponding elements in each of the several views. However, it is to be understood that disclosed aspects of the surgical stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

The disclosure is directed to a surgical stapling device that includes an elongate body that supports a tool assembly including a cartridge assembly having a replaceable staple cartridge. The staple cartridge includes a lockout member and an actuation sled assembly that includes an actuation sled and a knife. The knife is configured to disable the lockout member when the actuation sled assembly is in a sled retracted position and to prevent firing of the stapling device when the actuation sled assembly is not positioned in a proximal portion of the staple cartridge or when a staple cartridge is not present in the cartridge assembly.

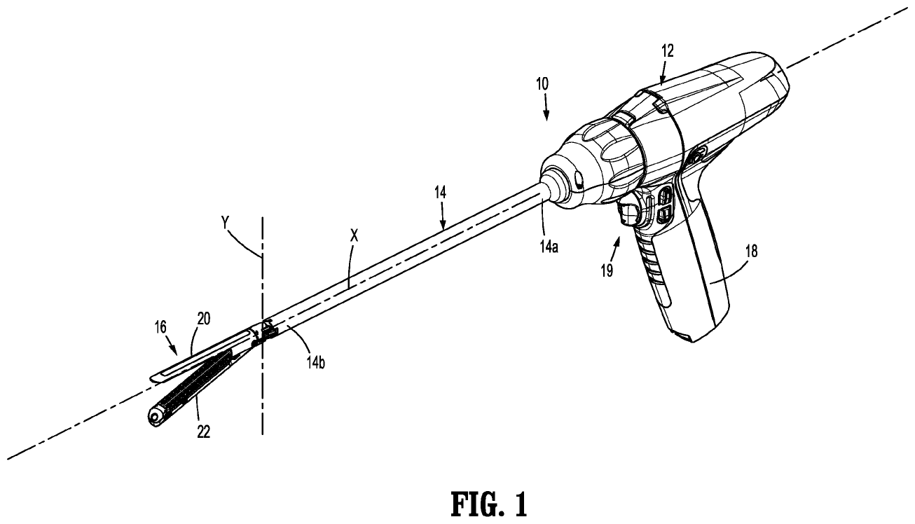

FIG. 1 illustrates a surgical stapling device shown generally as stapling device 10 that includes a handle assembly 12, an adapter assembly 14, and a tool assembly 16. The handle assembly 12 is powered and includes a stationary handgrip 18 and an actuation button or buttons 19 that are operable to actuate various functions of the tool assembly 16 via the adapter assembly 14, i.e., approximation of the tool assembly 16, and stapling and cutting of tissue. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the advantages of this disclosure are suitable for use with manually powered surgical stapling devices as well as robotically controlled stapling devices. U.S. Pat. No. 5,865,361 describes a stapling device that includes exemplary aspects of a manually powered stapling device.

The adapter assembly 14 defines a longitudinal axis “X” and includes a proximal portion 14a and a distal portion 14b. The proximal portion 14a of the adapter assembly 14 is coupled to the handle assembly 12, and the distal portion 14b of the adapter assembly 14 is coupled to the tool assembly 16. In aspects of the disclosure, the tool assembly 16 is pivotably coupled to the distal portion 14b of the adapter assembly 14 to facilitate articulation of the tool assembly 16 in relation to the adapter assembly 14 about an articulation axis “Y”.

The tool assembly 16 includes an anvil 20 and a cartridge assembly 22. In aspects of the disclosure, the anvil 20 is fixedly secured to the distal portion 14b of the adapter assembly 14 and the cartridge assembly 22 is pivotably supported in relation to the anvil 20 to facilitate movement of the tool assembly 16 between an unclamped position (FIG. 1) and a clamped position (FIG. 5). It is envisioned that the cartridge assembly 22 could be fixedly secured to the distal portion 14b of the adapter assembly 14 and the anvil 20 could be pivotably supported in relation to the cartridge assembly 22 to facilitate movement of the tool assembly 16 between the unclamped position and the clamped position.

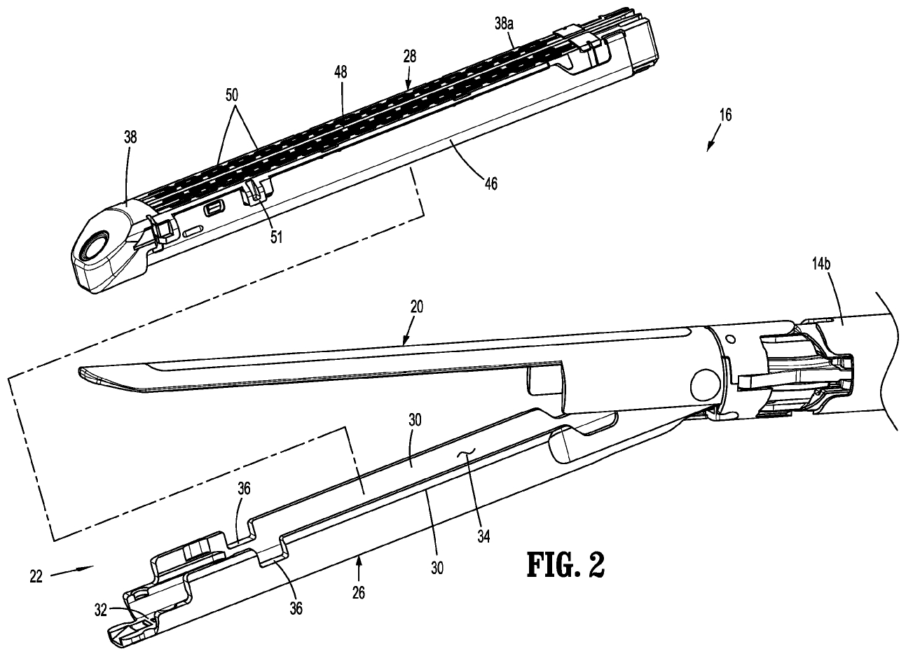

FIG. 2 illustrates the cartridge assembly 22 which includes a channel 26 and a staple cartridge 28. The channel 26 includes side walls 30 and a bottom wall 32 that define a cavity 34. The staple cartridge 28 is removably received within the cavity 34 of the channel 26 and is replaceable to facilitate reuse of the stapling device 10. Each of the side walls 30 of the channel 26 has an upper end spaced from the bottom wall 32 that defines a recess 36.

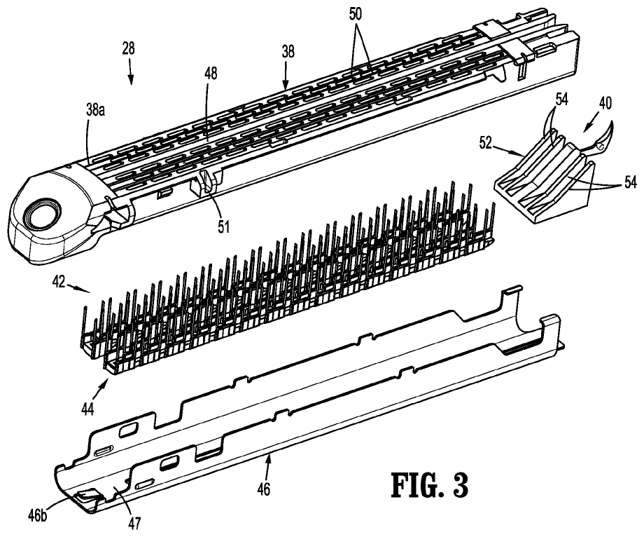

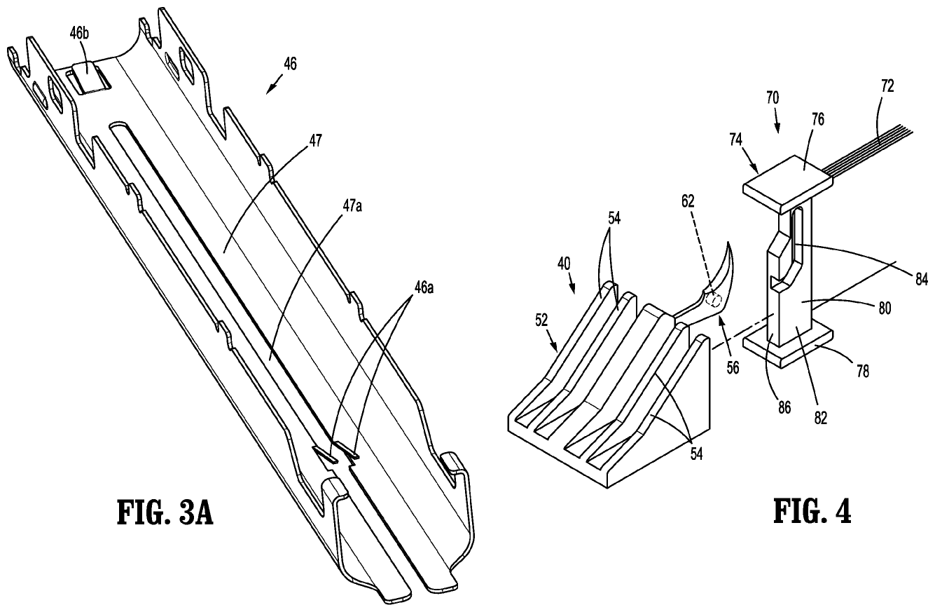

FIGS. 2-3A illustrate the staple cartridge 28 of the cartridge assembly 22 which includes a cartridge body 38, an actuation sled assembly 40, staples 42, pushers 44, and a staple guard 46. The cartridge body 38 includes a tissue engaging surface 38a and defines a knife slot 48 and staple receiving pockets 50 that extend through the tissue engaging surface 38a. The staple receiving pockets 50 are positioned on opposite sides of the knife slot 48 and can be aligned in two or more rows. In other aspects of the disclosure, the knife slot 48 is positioned along a central longitudinal axis of the cartridge body 38. However, it is envisioned that the knife slot 48 could be offset from the central longitudinal axis of the cartridge body 38 and the number of rows of staple receiving pockets 50 on opposite sides of the knife slot 48 can be different. The cartridge body 38 includes laterally extending protrusions 51 (only one is shown) that are received within the recesses 36 (FIG. 2) of the channel 26 to properly position the staple cartridge 28 within the cavity 34 of the channel 26.

The staples 42 and the pushers 44 are received within the staple receiving pockets 50 of the cartridge body 38, and the staple guard 46 is secured to the bottom of the cartridge body 38 opposite to the tissue engaging surface 38a to retain the staples 42 and pushers 44 within the cartridge body 38. The staple guard 46 includes a bottom wall 47 that defines a knife slot 47a and includes locking members in the form of resilient tabs 46a, 46b (FIG. 3A) that extend upwardly from the bottom wall 47 of the staple guard 46 into the cartridge body 38. The knife slot 47a is longitudinally aligned with the knife slot 48 in the cartridge body 38. The resilient tabs 46a are positioned adjacent a proximal portion of the knife slot 47a and are positioned on opposite sides of the knife slot 47. The resilient tab 46b is positioned distally of the knife slot 47 and is longitudinally aligned with the knife slot 47. In aspects of the disclosure, the resilient tabs 46a, 46b are integrally formed with the staple guard 46 and are secured in cantilevered fashion to the bottom wall 47 of the staple guard 46 such that the resilient tabs 46a, 46b are angled upwardly into the cartridge body 38. In aspects of the disclosure, the resilient tabs 46a are secured to the bottom wall 47 of the staple guard 46 at their distal ends and the resilient tab 46b is secured to the bottom wall 47 of the staple guard 46 at its proximal end.

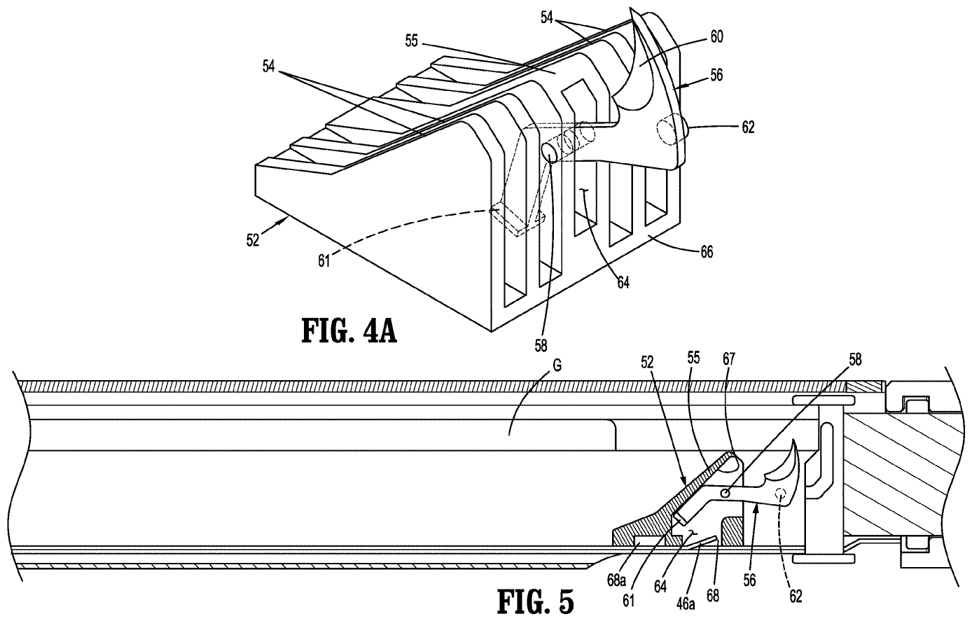

FIGS. 4 and 4A illustrate the actuation sled assembly 40 which is movable through the cartridge body 38 (FIG. 3) from a sled retracted position to a sled advanced position and includes an actuation sled 52 having angled cam members 54 and a knife 56. The cam members 54 are positioned to sequentially engage the pushers 44 (FIG. 3) as the actuation sled assembly 40 moves through the cartridge body 38 to lift the pushers 44 within the staple receiving pockets 50 of the cartridge body 38 and eject the staples 42 from the cartridge body 38. The actuation sled 52 includes a central body portion 55 that is positioned between the cam members 54 and is received within the knife slot 48 of the cartridge body 38 as the actuation sled assembly 40 moves from the sled retracted position towards the sled advanced position.

The knife 56 is pivotally secured to the central body portion 55 of the actuation sled 52 by a pivot member 58. In aspects of the disclosure, the pivot member 58 can be integrally formed with the knife 56. Alternately, the pivot member 58 can be in the form of a pin that is inserted through the knife 56 and through the central body portion 55 of the actuation sled 52 of the actuation sled assembly 40. The knife 56 includes a first portion positioned proximally of the pivot member 58 and a second portion positioned distally of the pivot member 58. The first portion of the knife 56 includes a cutting edge 60 that extends upwardly from the pivot member 58 towards the anvil 20 (FIG. 2) and includes or supports a cam member 62. In aspects of the disclosure, the cam member 62 extends from one side of the first portion of the knife 56 towards the cam members 54 of the actuation sled 52. The second portion of the knife 56 extends downwardly from the pivot member 58 towards the staple guard 46 and includes an engagement member 61. In aspects of the disclosure, the engagement member 61 defines a T-shape with the second portion of the knife 56 and is positioned to engage the resilient tabs 46a as described in further detail below.

In aspects of the disclosure, the central body portion 55 of the actuation sled 52 defines a cavity 64 (FIG. 4A) and the knife 56 is pivotally supported within the cavity 64 and movable between a lowered position and a raised position. The actuation sled 52 includes a base portion 66 that defines an opening 68 and a recess 68a (FIG. 5). The opening 68 communicates with the cavity 64 in the central body portion 55 of the actuation sled 52 and is dimensioned to receive the resilient tabs 46a of the staple guard 46 of the staple cartridge 28 when the actuation sled assembly 40 is in the sled retracted position to prevent advancement of the actuation sled assembly 40. The recess 68a is dimensioned to receive the resilient tab 46b of the staple guard 46 when actuation sled assembly 40 is in the sled advanced position to retain the actuation sled assembly 40 in the sled advanced position. The cavity 64 is defined in part by an upper stop surface 67 that is spaced from the base portion 66 of the actuation sled 52 and engages the second portion of the knife 56 when the knife 56 is in the lowered position.

FIG. 4 illustrates a drive assembly 70 of the stapling device 10 which includes a flexible drive beam 72 and a working member 74. The flexible drive beam 72 includes stacked sheets that have distal ends secured to the working member 74. The working member 74 has an I-beam configuration and includes a first beam 76, a second beam 78, and a vertical strut 80 that secures the first and second beams 76, 78 together at a fixed distance from each other. The vertical strut 80 has a side wall 82 and a distal surface 86. The distal surface 86 is positioned to engage the proximal portion of the actuation sled 52 when the stapling device 10 is fired to advance the actuation sled assembly 40 from the sled retracted position to the sled advanced position. The side wall 82 of the vertical strut 80 defines a cam slot 84. In aspects of the disclosure, the cam slot 84 has a J-shaped configuration. The drive assembly 70 is movable through the cartridge body 38 (FIG. 2) from a drive retracted position, to a drive clamped position, to a drive advanced position to advance the actuation sled assembly 40 (FIG. 5) through the cartridge body 38 from the sled retracted position to the sled advanced position. As the drive assembly 70 moves through the cartridge body 38 from the drive retracted position to the drive clamped position, the vertical strut 80 of the working member 74 begins to move through the knife slot 48 (FIG. 3) of the cartridge body 38, and the first and second beams 76, 78 of the working member 74 engage the anvil 20 and the cartridge assembly 22, respectively, to pivot the cartridge assembly 22 towards the anvil 20 and move the tool assembly 16 from the unclamped position to the clamped position. In the clamped position, the upper and lower beams 76, 78 of the working member 74 of the drive assembly 70 define a maximum tissue gap between the anvil 20 and the cartridge assembly 22 during firing.

FIG. 5 illustrates the tool assembly 16 of the stapling device 10 (FIG. 1) with the tool assembly 16 in the clamped position prior to firing of the stapling device 10. In the clamped position, the working member 74 of the drive assembly 70 is spaced proximally of the actuation sled assembly 40 and the actuation sled assembly 40 is in the sled retracted position. In the sled retracted position, the resilient tabs 46a extend through the opening 68 in the staple guard 46 and engage an inner surface of the actuation sled 52 to retain the actuation sled assembly 40 in the sled retracted position. In the drive clamped position of the drive assembly 70, the knife 56 is in the lowered position with the cutting edge 60 of the knife 56 recessed within the knife slot 48 of the cartridge body 38, and the second portion of the knife 56 is engaged with the stop surface 67 of the central body portion 55 of the actuation sled 52 to prevent rotation of the knife 56 in a clockwise direction as viewed in FIG. 5.

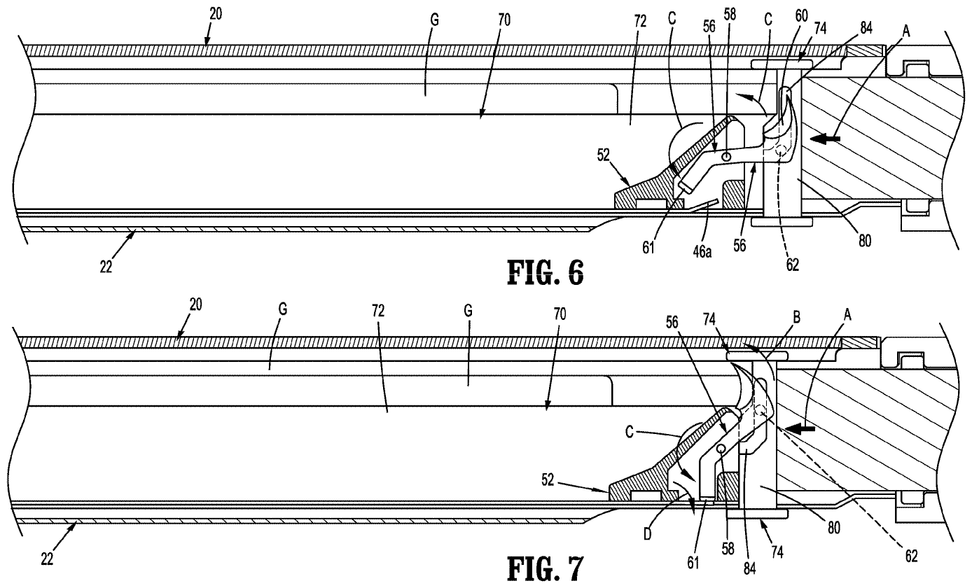

FIGS. 6 and 7 illustrate the tool assembly 16 of the stapling device 10 as the drive assembly 70 moves in the direction of arrow “A” from the drive clamped position towards the drive advanced position. As the drive assembly 70 moves from the drive clamped position towards the drive advanced position during firing of the stapling device 10 (FIG. 1), the cam member 62 on the knife 56 of the actuation sled assembly 40 is received within the cam slot 84 formed in the vertical strut 80 of the working member 74 of the drive assembly 70. When this occurs, continued advancement of the drive assembly 70 pivots the second portion of the knife 56 about a transverse axis defined by the pivot member 58 upwardly in the direction of arrow “B” in FIG. 7 from the lowered position in which the cutting edge 60 is recessed within the knife slot 48 of the cartridge body 38 of the staple cartridge 28 to the raised position in which the cutting edge 60 of the knife 56 is positioned above the tissue engaging surface 38a of the cartridge body 38 and extends across a gap “G” defined between tissue engaging surface 38a of the cartridge body 38 of the staple cartridge 28 and the anvil 20 (FIG. 2). As the first portion of the knife 56 pivots upwardly, the second portion of the knife 56 pivots downwardly about the transverse axis defined by the pivot member 58 in the direction indicated by arrow “C” such that the engagement member 61 engages the resilient tabs 46a and moves the resilient tabs 46a downwardly towards the channel 26 of the cartridge assembly 22 through the opening 68 in the actuation sled 52 from a locked position to an unlocked position. In the unlocked position of the resilient tabs 46a, the resilient tabs 46a are no longer in alignment with the actuation sled 52. In this position, the actuation sled assembly 40 can move from the sled retracted position towards the sled advanced position to advance the distal surface 86 of the vertical strut 80 of the working member 74 of the drive assembly 70 into engagement with the proximal surface of the actuation sled 52.

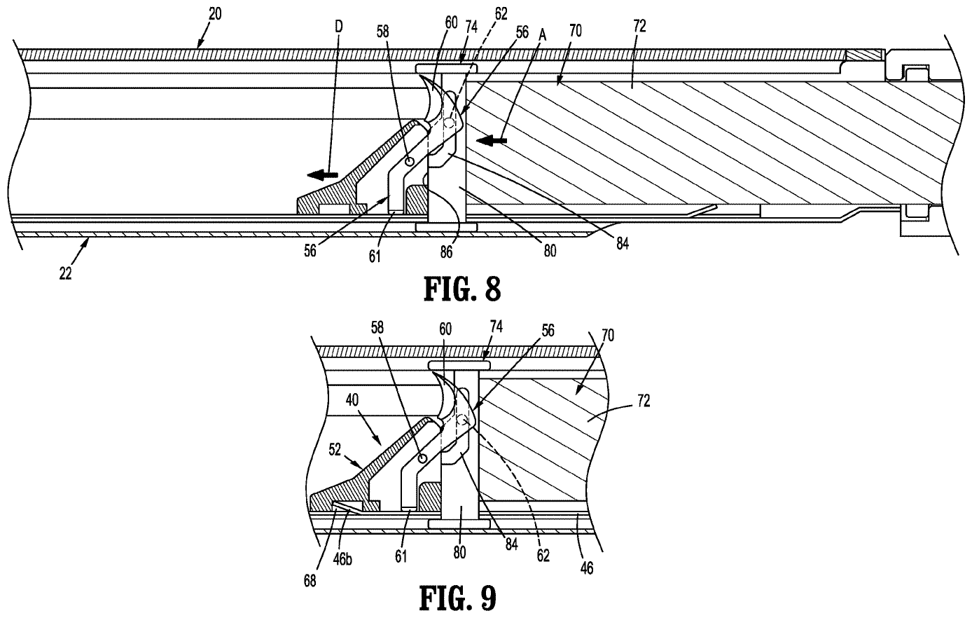

FIGS. 8 and 9 illustrate the tool assembly 16 of the stapling device 10 as the drive assembly 70 moves further in the direction of arrow “A” during the firing stroke of the stapling device 10 (FIG. 1) towards the drive advanced position. Once the resilient tabs 46a are moved from the locked position to the unlocked position, the actuation sled assembly 40 is moved through the staple cartridge 28 by the working member 74 of the drive assembly 70 in the direction indicated by arrow “D” from the sled retracted position towards the sled advanced position with the knife 56 retained in the raised position via receipt of the cam member 62 of the knife 56 within the cam slot 84 of the working member 74. When the actuation sled assembly 40 reaches the sled advanced position, the resilient tab 46b of the staple guard 46 of the staple cartridge 28 is received within the recess 68a in the base portion 66 of the actuation sled 52 to retain the actuation sled assembly 40 in the sled advanced position.

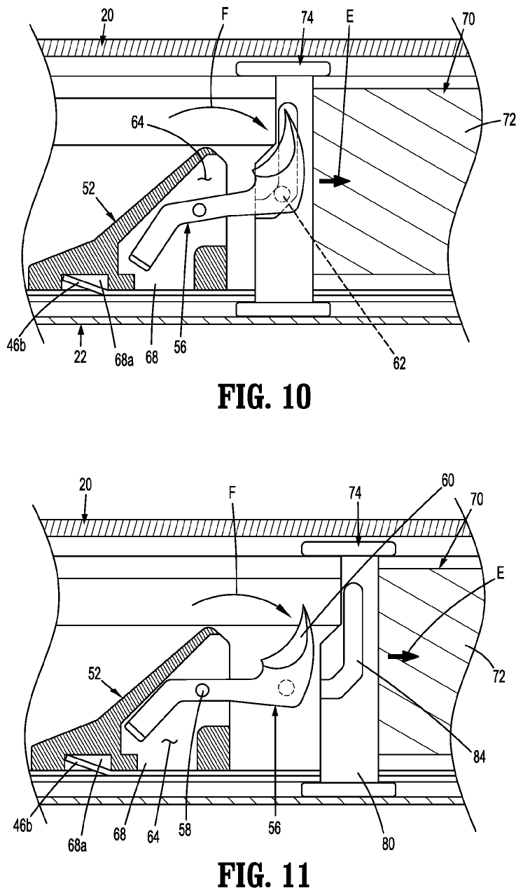

FIGS. 10 and 11 illustrate the drive assembly 70 as the drive assembly 70 moves from the drive advanced position towards the drive retracted position after the stapling device 10 has been fired (FIG. 1). When the drive assembly 70 is moved from the drive advanced position back towards the drive retracted position, the working member 74 moves in the direction of arrow “E” while the actuation sled assembly 40 is retained in the sled advanced position by the resilient tab 46b of the staple guard 46. As the working member 74 moves in the direction of arrow “E”, the cam member 62 of the knife 56 moves through the cam slot 84 in the vertical strut 80 of the working member 74 to pivot the knife 56 in the direction of arrow “F” from the raised position back towards the lowered position. The drive assembly 70 disengages from the knife 56 of the actuation sled assembly 40 and returns to the drive retracted position. With the drive assembly 70 is in the drive retracted position after firing and the actuation sled assembly 40 is in the sled advanced position, the resilient tabs 46a remain in their locked positions to prevent readvancement of the drive assembly 70.

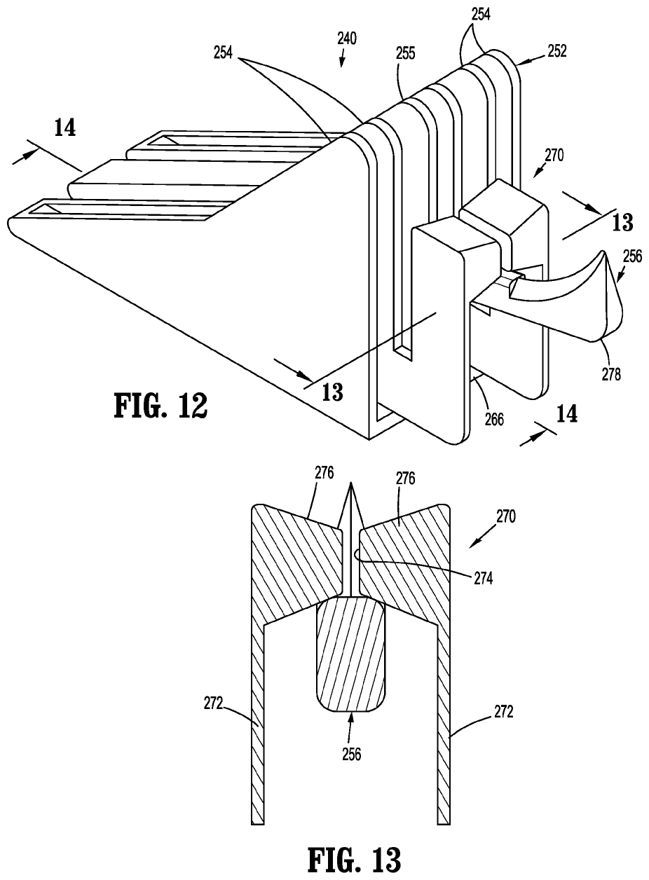

FIGS. 12-19 illustrate an alternate version of the tool assembly shown generally as tool assembly 216 (FIG. 15) which includes a cartridge assembly 220 having a staple cartridge 28 (FIG. 2) with an actuation sled assembly 240. It is noted that the staple cartridge of the tool assembly 216, except for the changes to the actuation sled assembly described herein, is as described above regarding staple cartridge 28. FIGS. 13-15 illustrate the actuation sled assembly 240 which includes an actuation sled 252 and a knife 256 that is pivotably coupled to the actuation sled 252. As described above regarding the actuation sled 52 (FIG. 4A), the actuation sled 252 includes angled cam members 254 that are positioned to sequentially engage the pushers 44 of the staple cartridge 28 (FIG. 3) as the actuation sled assembly 240 moves through the cartridge body 38 of the staple cartridge 28 (FIG. 3) to lift the pushers 44 within the staple receiving pockets 50 and eject the staples 42 (FIG. 3) from the cartridge body 38. The actuation sled 252 includes a central body portion 255 that is positioned between the cam members 254 and is received within the knife slot 48 (FIG. 3) of the cartridge body 38 as the actuation sled assembly 240 moves from the sled retracted position towards the sled advanced position.

The actuation sled 252 also includes a retention member 270 that extends from a proximal portion of the actuation sled 252. In aspects of the disclosure, the retention member 270 includes two resilient arms 272 that are formed on the proximal portion of the actuation sled 252 and are spaced from each other to define a slot 274. In aspects of the disclosure, the resilient arms 272 include protrusions 276 that are spaced to define the slot 274.

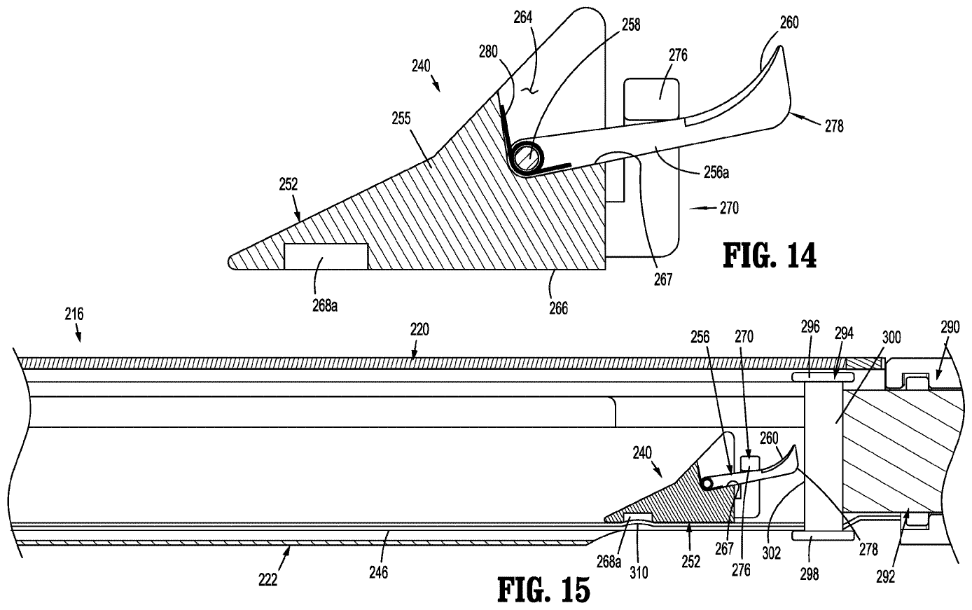

The knife 256 is pivotally secured to the central body portion 255 of the actuation sled 252 by a pivot member 258 (FIG. 14) and is movable between a lowered position and a raised position. In aspects of the disclosure, the pivot member 258 can be integrally formed with the knife 256. Alternately, the pivot member 258 can be in the form of a pin that is inserted through the knife 256 and through the central body portion 255 of the actuation sled 252 of the actuation sled assembly 240. The knife 256 includes a distal portion secured to the pivot member 258 and a proximal portion that includes a cutting edge 260 that extends upwardly from the pivot member 258 towards the anvil 220 (FIG. 15). In the lowered position, the knife 256 is positioned below the protrusions 276 of the retention member 270 to retain the knife 256 in the lowered position. The knife 256 must pass through the slot 274 in the retention member 270 to move between the lowered and raised positions. The knife 256 includes a curved surface 278 that is positioned at the proximal end of the knife 256.

In aspects of the disclosure, the central body portion 255 of the actuation sled 252 defines a cavity 264 (FIG. 14) and the knife 256 is pivotally supported within the cavity 264 between the lowered position and the raised position. The actuation sled 252 includes a base portion 266 (FIG. 12) that defines a recess 268a (FIG. 14) that is dimensioned to receive a locking member in the form of a resilient tab 246a (FIG. 19) of the staple guard 246 of the staple cartridge 28 (FIG. 3) when actuation sled assembly 240 is in the sled advanced position to retain the actuation sled assembly 240 in the sled advanced position. The cavity 264 in the central body portion 255 of the actuation sled 252 is defined in part by a support surface 267 (FIG. 14) that supports the knife 256 when the knife 256 is in the lowered position. The support surface 267 is positioned between the base portion 266 of the actuation sled 252 and the pivot member 258. In some aspects of the disclosure, the support surface 267 slopes upwardly in a proximal direction. In the lowered position, the knife 256 includes a central portion 256a (FIG. 15) that is engaged with the protrusions 276 of the retention member 270 such that the knife 256 is retained in the lowered position. The central portion 256a of the knife 256 is aligned with the slot 274 in the retention member 270 and has a width that is greater than the width of the slot 274.

In some aspects of the disclosure, the knife 256 is urged to the lowered position by a biasing member 280. In certain aspects of the disclosure, the biasing member 280 includes a torsion spring that is received about the pivot member 258 and urges the knife 256 in a clockwise direction as viewed in FIG. 14 against the support surface 267.

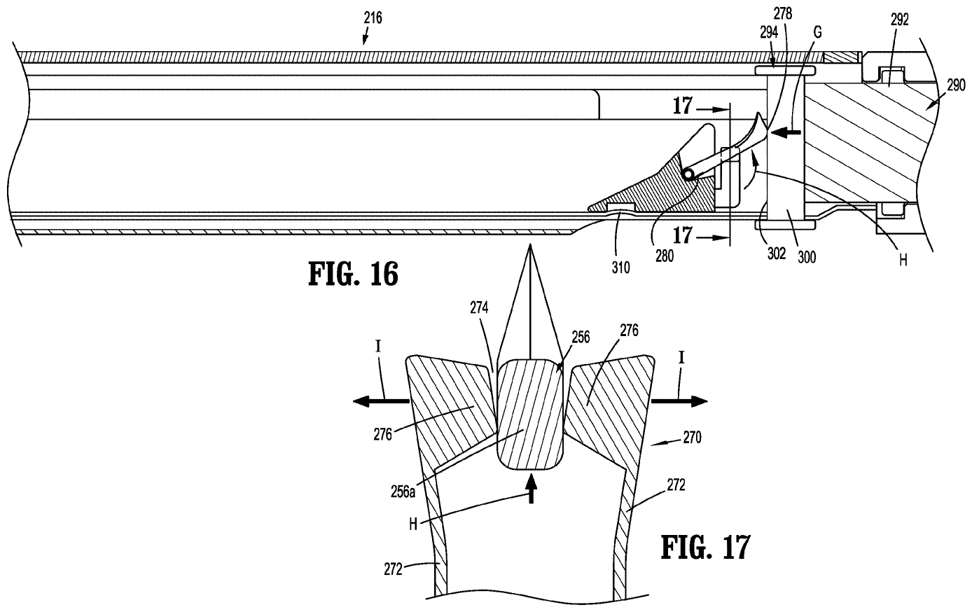

FIG. 16 illustrates an alternate version of the drive assembly of the stapling device 10 (FIG. 1) shown generally as drive assembly 290. The drive assembly 290 includes a flexible drive beam 292 and a working member 294. The flexible drive beam 292 includes stacked sheets that have distal ends secured to the working member 294. The working member 294 has an I-beam configuration and includes a first beam 296, a second beam 298, and a vertical strut 300 that secures the first and second beams 296, 298 together at a fixed distance from each other. The vertical strut 300 has a distal surface 302 that is positioned to engage the proximal portion of the actuation sled 252 when the stapling device 10 (FIG. 1) is fired to advance the actuation sled assembly 240 from the sled retracted position to the sled advanced position and eject the staples 42 (FIG. 2) from the cartridge body 38 of the staple cartridge 28 (FIG. 3). As the drive assembly 290 moves through the cartridge body 38 (FIG. 3) from the drive retracted position to the drive clamped position, the vertical strut 300 of the working member 294 begins to move through the knife slot 48 (FIG. 2) of the cartridge body 38, and the first and second beams 296, 298 of the working member 294 engage the anvil 220 and the cartridge assembly 222 (FIG. 2), respectively, to pivot the cartridge assembly 222 towards the anvil 220 to move the tool assembly 216 from the unclamped position to the clamped position.

FIG. 15 illustrates the tool assembly 216 in the clamped position prior to firing of the stapling device with the drive assembly 290 in the drive clamped position. In the clamped position, the working member 294 of the drive assembly 290 is spaced proximally of the actuation sled assembly 240 and the actuation sled assembly 240 is in the sled retracted position. In the sled retracted position, a detent 310 formed on the staple guard 246 of the staple cartridge 28 (FIG. 3) is received within the recess 268a of the actuation sled 252 to retain the actuation sled assembly 240 in the sled retracted position until a pre-determined force in the distal direction is applied to the actuation sled assembly 240. The knife 256 is in the lowered position with the cutting edge 260 of the knife 256 recessed within the knife slot 48 (FIG. 2) of the cartridge body 38. The knife 256 is supported on the support surface 267 beneath the protrusions 276 of the retention member 270 to retain the knife 256 in the lowered position.

FIGS. 16 and 17 illustrate the tool assembly 216 of the stapling device 10 (FIG. 1) as the drive assembly 290 is advanced in the direction of arrow “G” in FIG. 17 from the drive retracted position towards the drive advanced position during firing of the stapling device 10 (FIG. 1). When the drive assembly 290 is advanced from the drive clamped position towards the drive retracted position, the distal surface 302 of the vertical strut 300 engages the curved proximal surface 278 of the knife 256 and pivots the knife 252 upwardly in the direction indicated by arrow “H” to move the knife 256 from the lowered position to the raised position. As the knife 256 pivots from the lowered position towards the raised position, the detent 310 retains the actuation sled assembly 240 in the sled retracted position as the knife 256 passes through the slot 274 (FIG. 17) in the retention member 270 and is moved to the raised position. As the knife 256 passes through the slot 274, the knife 256 engages the protrusions 276 to urge the resilient arms 272 outwardly in the direction indicated by arrows “I” in FIG. 17 to allow the central portion 256a of the knife 256 to pass through the slot 274 in the retention member 270 to the raised position. When the knife 256 passes through the slot 274 and moves to the raised position, the resilient arms 272 return to their unbiased state and the distal surface 302 of the vertical strut 300 of the drive assembly 290 retains the knife 256 in the raised position.

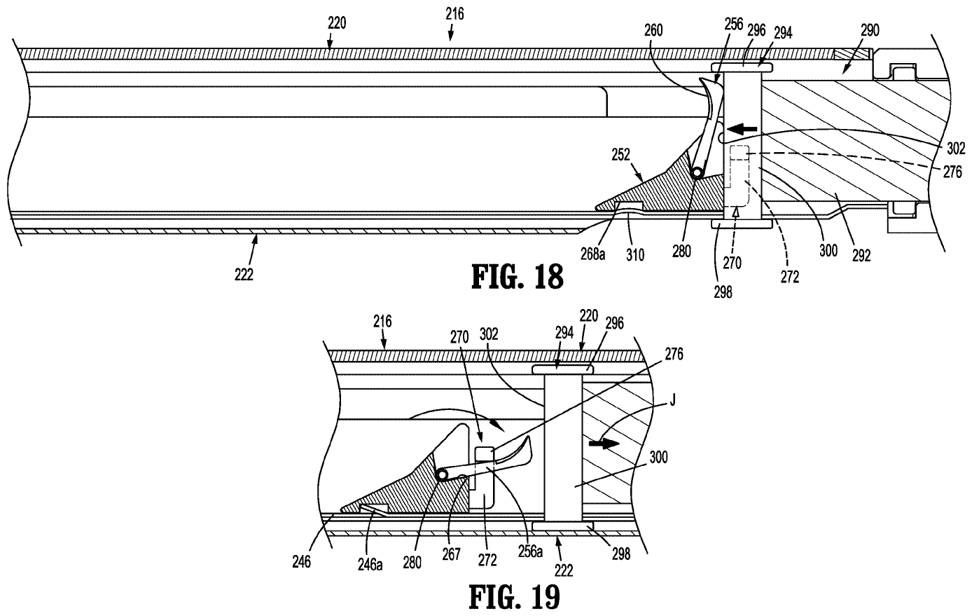

FIG. 18 illustrates the knife 256 in the raised position with the distal surface of the vertical strut 300 engaged with the proximal surface of the actuation sled 252. When the drive assembly 290 is advanced to a position to move the knife 256 to the raised position, the vertical strut 300 of the drive assembly 290 moves through the slot 274 of the retention member 270 and engages the central body portion 255 of the actuation sled 252. Further advancement of the drive assembly 290 from this position advances the actuation sled assembly 240 through the cartridge body 38 (FIG. 3) to eject the staples 42 (FIG. 3) from the cartridge body 38 and cut tissue clamped within the tool assembly 216.

FIG. 19 illustrates the actuation sled assembly 240 in the sled advanced position as the drive assembly 290 is retracted from the drive advanced position towards the drive retracted position in the direction indicated by arrow “J”. When the drive assembly 290 is returned from the drive advanced position to the drive retracted position, the actuation sled assembly 240 is retained in the sled advanced position by the resilient tab 246a of the staple guard 246 and the biasing member 280 returns the knife 256 though the slot 274 of the retention member 270 to the lowered position.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A tool assembly comprising:
an anvil; and
a cartridge assembly including a channel and a staple cartridge, the channel defining a cavity, the staple cartridge releasably supported within the cavity and including:
a cartridge body having a tissue engaging surface and defining a knife slot and staple receiving pockets that extend through the tissue engaging surface;
staples received within the staple receiving pockets of the cartridge body;
an actuation sled assembly supported within the cartridge body and including an actuation sled and a knife, the knife pivotably supported on the actuation sled by a pivot member and movable between a lowered position and a raised position, the actuation sled assembly movable within the cartridge body from a sled retracted position to a sled advanced position to eject the staples from the cartridge body; and
a locking member movable between a locked position and an unlocked position, wherein in the locked position, the locking member is engaged with the actuation sled assembly when the actuation sled assembly is in the sled retracted position to retain the actuation sled assembly in the sled retracted position, the locking member movable from the locked position to the unlocked position in response to movement of the knife from the lowered position to the raised position.

2. The tool assembly of claim 1, further including a drive assembly having a working member, the working member having a first beam, a second beam, and a vertical strut connecting the first beam to the second beam, the drive assembly movable between a drive retracted position and a drive advanced position.

3. The tool assembly of claim 2, wherein the vertical strut defines a cam slot.

4. The tool assembly of claim 3, wherein the knife includes a cam member that is received within the cam slot when the drive assembly is moved towards the drive advanced position to pivot the knife from the lowered position to the raised position.

5. The tool assembly of claim 1, wherein the knife includes a cutting edge positioned proximally of the pivot member and an engagement member positioned distally of the pivot member.

6. The tool assembly of claim 5, wherein the engagement member is positioned to engage the locking member when the knife is in the raised position to move the locking member to the unlocked position.

7. The tool assembly of claim 6, wherein the locking member includes first resilient tabs, the first resilient tabs positioned to prevent advancement of the drive assembly from the drive retracted position to the drive advanced position when the actuation sled assembly is in the sled advanced position.

8. The tool assembly of claim 7, wherein the actuation sled of the actuation sled assembly includes a base portion that defines an opening, and the first resilient tabs are received within the opening in the locked position.

9. The tool assembly of claim 8, wherein the staple cartridge includes a staple guard that is secured to a bottom wall of the cartridge body to retain the staples within the cartridge body, the staple guard having a bottom wall defining a knife slot, the knife slot of the staple guard aligned with the knife slot of the cartridge body.

10. The tool assembly of claim 9, wherein the first resilient tabs extend in cantilevered fashion from the bottom wall of the staple guard.

11. The tool assembly of claim 9, wherein the actuation sled defines a recess and the staple guard includes a second resilient tab positioned on a distal portion of the bottom wall of the staple guard, the second resilient tab positioned to be received in the recess of the actuation sled to retain the actuation sled assembly in the sled advanced position.

12. The tool assembly of claim 5, wherein the engagement member is formed on a distal end of the knife and has a T-shaped configuration.

13. The tool assembly of claim 4, wherein the cam slot of the vertical strut of the working member has a J-shaped configuration.

14. A stapling device comprising:
a handle assembly;
an adapter assembly having a distal portion and a proximal portion, the proximal portion of the adapter assembly coupled to the handle assembly; and
a tool assembly coupled to the distal portion of the adapter assembly, the tool assembly including:
an anvil; and
a cartridge assembly including a channel and a staple cartridge, the channel defining a cavity, the staple cartridge releasably supported within the cavity and including:
a cartridge body having a tissue engaging surface and defining a knife slot and staple receiving pockets that extend through the tissue engaging surface;
staples received within the staple receiving pockets of the cartridge body;
an actuation sled assembly supported within the cartridge body and including an actuation sled and a knife, the knife pivotably supported on the actuation sled by a pivot member and movable between a lowered position and a raised position, the actuation sled assembly movable within the cartridge body from a sled retracted position to a sled advanced position to eject the staples from the cartridge body; and
a locking member movable between a locked position and an unlocked position, wherein in the locked position, the locking member is engaged with the actuation sled assembly when the actuation sled assembly is in the sled retracted position to retain the actuation sled assembly in the sled retracted position, the locking member movable from the locked position to the unlocked a position in response to movement of the knife from the lowered position to the raised position.

15. The stapling device of claim 14, further including a drive assembly having a working member, the working member having a first beam, a second beam, and a vertical strut connecting the first beam to the second beam, the vertical strut defining a cam slot, the drive assembly movable between a drive retracted position and a drive advanced position.

16. The stapling device of claim 15, wherein the knife includes a cam member that is received within the cam slot when the drive assembly is moved towards the drive advanced position to pivot the knife from the lowered position to the raised position.

17. The stapling device of claim 15, wherein the locking member includes first resilient tabs, the first resilient tabs positioned to prevent advancement of the drive assembly from the drive retracted position to the drive advanced position when the actuation sled assembly is in the sled advanced position.

18. The stapling device of claim 17, wherein the staple cartridge includes a staple guard that is secured to a bottom wall of the cartridge body to retain the staples within the cartridge body, the staple guard having a bottom wall defining a knife slot, the knife slot of the staple guard aligned with the knife slot of the cartridge body, and the resilient tabs are formed with the bottom wall of the staple guard.

19. The stapling device of claim 18, wherein the actuation sled defines a recess and the staple guard includes a second resilient tab positioned on a distal portion of the bottom wall of the staple guard, the second resilient tab positioned to be received in the recess of the actuation sled to retain the actuation sled assembly in the sled advanced position.

20. A tool assembly comprising:
an anvil; and
a cartridge assembly including a channel and a staple cartridge, the channel defining a cavity, the staple cartridge releasably supported within the cavity and including:
a cartridge body having a tissue engaging surface and defining a knife slot and staple receiving pockets that extend through the tissue engaging surface;
staples received within the staple receiving pockets of the cartridge body; and
an actuation sled assembly supported within the cartridge body and including an actuation sled and a knife, the knife pivotably supported on the actuation sled by a pivot member and movable between a lowered position and a raised position, the actuation sled assembly movable within the cartridge body from a sled retracted position to a sled advanced position to eject the staples from the cartridge body, the actuation sled supporting a retention member that extends from a proximal portion of the actuation sled and includes spaced resilient arms, each of the spaced resilient arms having a protrusion that is positioned to engage the knife when the knife is in the lowered position to retain the knife in the lowered position.
